(19) United States
(12) Patent Application Publication
(10) Pub. No.: US 2025/0359871 A1
(43) Pub. Date: Nov. 27, 2025

(54) LOOPED TISSUE COMPRESSION DEVICES, SYSTEMS, AND METHODS

(71) Applicant: Boston Scientific Scimed, Inc., Maple Grove, MN (US)

(72) Inventors: Gonzalo Jose Saenz Villalobos, Alajuela (CR); Oscar Segura Elizondo, San Jose (CR); Gabriela Gamboa Bastos, San Jose (CR); Carolina Padilla Castro, San Jose (CR)

(73) Assignee: Boston Scientific Scimed, Inc., Maple Grove, MN (US)

(21) Appl. No.: 19/217,704

(22) Filed: May 23, 2025

Related U.S. Application Data
(60) Provisional application No. 63/651,501, filed on May 24, 2024.

(51) Int. Cl. A61B 17/12

(52) U.S. Cl. CPC A61B 17/12013

(57) ABSTRACT

A compression device having an elongated compressive element which can be looped into and adjusted to different desirable sizes around target tissue. The compressive element may be a simple suture string or the like having a distal portion looped around the target tissue and extending to a proximal end positioned outside the patient's body and graspable and manipulable by a medical professional to adjust the looped distal portion. A cinch device may be preloaded on and delivered with the compressive element to target tissue. The cinch device may be coupled with the compressive element in a manner which may facilitate maintenance of the looped configuration of the distal portion of the compressive element and/or to hold the distal portion of the compressive element in a final looped configuration applying the desired compressive force to the target tissue surrounded by the compressive element.

### CROSS-REFERENCE TO RELATED APPLICATIONS

This application claims the benefit of priority under 35 U.S.C. § 119 to U.S. Provisional Application No. 63/651,501, filed May 24, 2024, the entire disclosure of which is hereby incorporated by reference herein for all purposes.

### FIELD

The present disclosure relates generally to medical devices, systems, and methods for applying compressive forces around (e.g., surrounding) tissue. More particularly, the present disclosure relates to medical devices, systems, and methods for encircling tissue with an elongate element to apply compressive forces circumferentially around the tissue.

### BACKGROUND

The positioning and tightening of a loop of material around tissue to encircle the tissue and to apply a compressive (generally radially-inwardly) force to the tissue is a common method for treating various conditions, such as to control and/or prevent bleeding, to ligate tissue (e.g., polyps or tumors), and/or to close an open area of tissue. In the case of tissue ligation and removal, the loop provides a mechanical compression over the tissue (e.g., pedunculated polyp) to decrease the risk of bleeding post tissue resection. Commercially available devices include the Endoloop™ Ligature sold by Ethicon US, LLC. However, common commercially-available devices do not afford variability in the loop size, and some commercially-available devices only allow for one-way tightening without the option to readjust the size of the loop. Some commercially-available devices require further elements, such as tissue clips, to maintain a loop in place around tissue so that the loop size may then be reduced to apply compressive force to the clipped tissue. Other tools, not sold as a ligation system, may also be used, such as a nylon-based string, to loop around tissue to apply mechanical compression to the encircled tissue. However, without accompanying structures guiding the string and/or maintaining the position during application of force and/or maintaining the ultimate position of the string, use of such simplified tools may be challenging during a medical procedure. Alternatives to loops include staples for wound closure, and elastic ligation bands for cutting off blood supply to tissue to be removed/excised. However, such devices also have various limitations, such as the degree to which such devices may be opened to be placed around or otherwise to engage tissue, as well as the amount of compressive force which such devices may apply to tissue to achieve the intended procedural result. It is with respect to these and other considerations that the present improvements may be useful.

### SUMMARY

This Summary is provided to introduce, in simplified form, a selection of concepts described in further detail below in the Detailed Description. This Summary is not intended to necessarily identify key features or essential features of the claimed subject matter, nor is it intended as an aid in determining the scope of the claimed subject matter. One of skill in the art will understand that each of the various aspects and features of the present disclosure may advantageously be used separately in some instances, or in combination with other aspects and features of the disclosure in other instances, whether or not described in this Summary. No limitation as to the scope of the claimed subject matter is intended by either the inclusion or non-inclusion of elements, components, or the like in this Summary.

In accordance with various principles of the present disclosure, a system for applying compressive forces around target tissue includes a delivery device; and a compression device comprising a compressive element having a flexible distal portion configured to surround the target tissue and ending in a distalmost end, and a proximally-extending portion extending proximally from the distal portion to a proximal end positionable outside the patient's body to be movable by a medical professional with respect thereto to adjust the size of the distal portion surrounding the target tissue to adjust compressive force on the target tissue.

In some aspects, the system further includes a cinch device having a first component coupled to the compressive element for delivery therewith by the delivery device to the target tissue. In some aspects, the cinch device further includes a second component coupled to the delivery device for delivery therewith to the target tissue. In some aspects, the first component and the second component are movable between an unlocked configuration in which the first component and the second component are spaced apart from each other, and a locked configuration in which the first component and the second component are coupled together. In some aspects, the first component of the cinch device is coupled to a distalmost end of the compressive element and to a first component of the delivery device movable with respect to a second component of the delivery device to position the compressive element with respect to the target tissue; the second component of the delivery device is a tubular member; and the second component of the cinch device is a cinch collar coupled to a distal end of the tubular member. In some aspects, a lumen defined within the cinch collar communicates with a lumen defined within the tubular member, and the proximally-extending portion of the compressive element extends through the lumens of the cinch collar and of the tubular member. In some aspects, the proximally-extending portion of the compressive element extends from the lumen of the tubular member through an opening in the wall of the tubular member to extend to the proximal portion of the compressive element outside the patient's body. In some aspects, the system further includes a cutter within the tubular member and movable to cut the proximally-extending portion of the compressive element.

In some aspects, the distalmost end of the compressive element is coupled to a first component of the delivery device; a section of the proximally-extending portion of the compressive element is movably coupled to a second component of the delivery device; the first component of the delivery device is movable with respect to the second component of the delivery device to position the compressive element with respect to the target tissue; and the proximal end of the compressive element is movable with respect to the delivery device to move the section of the compressive element with respect to the second component of the delivery device to adjust the size of the distal portion of the compressive element surrounding the target tissue. In some aspects, the system further includes a cinch element coupled to the first component of the delivery device, with the distalmost end of the compressive element coupled to the cinch element; and a cinch collar coupled to the second component of the delivery device and movable with the second component of the delivery device and with respect to the first component of the delivery device to engage the cinch element to maintain a configuration of the distal portion of the compressive element.

In some aspects, the system further includes an outer tubular delivery element defining a lumen through which the compression device is delivered to a target site within the patient's body. In some aspects, the system further includes a tension adjuster mounted on the outer tubular delivery element, wherein the proximal end of the compressive element is operably engaged with the tension adjuster, and movement of the tension adjuster moves the compressive element with respect to the delivery device. In some aspects, the system further includes a fitment mounted on the outer tubular delivery element and having at least one gripper configured to hold the proximal end of the compressive element in place with respect to the delivery device.

In accordance with various principles of the present disclosure, a tissue compression device includes an elongated compressive element having a distalmost end, a flexible distal portion extending proximally from the distalmost end and arranged in a loop configuration, a proximally-extending portion extending proximally from the distal portion and terminating at a proximal end; and a cinch device having a first component coupled to the distalmost end of the elongated compressive element. In some aspects, the distalmost end of the elongated compressive element and the first component of the cinch device are deliverable together to a target site within a body with the proximally-extending portion of the elongated compressive element extending to the proximal end of the elongated compressive element positioned outside the patient. In some aspects, the proximal end of the elongated compressive element is manipulable to adjust the loop configuration of the flexible distal portion of the elongated compressive element.

In some aspects, the cinch device of the tissue compression device further includes a second component adapted to be delivered to the target site with the elongated compressive element and spaced apart from the first component of the cinch device such that the cinch device is delivered in an unlocked configuration. In some aspects, the first component and the second component of the cinch device are movable into engagement with each other to shift the cinch device into a locked configuration with respect to the elongated compressive element. In some aspects, the proximally-extending portion of the elongated compressive element extends through an opening in the second component of the cinch device to form the distal portion of the elongated compressive element into the loop configuration, and is movable through the opening to adjust the loop configuration.

In accordance with various principles of the present disclosure, a method of applying and retaining a compressive element around target tissue within a patient includes delivering a distal portion of an elongated compressive element to a target site within the patient with a proximal end of the elongated compressive element remaining outside the patient; surrounding target tissue with the distal portion of the elongated compressive element; and maintaining a distalmost end of the elongated compressive element adjacent the surrounded target tissue while moving a proximally-extending portion of the elongated compressive element to adjust the area enclosed by and within the distal portion.

In some aspects, the method further includes utilizing a first component of a delivery device used to deliver the distal portion of the elongated compressive element to maintain the distalmost end of the elongated compressive element in place with respect to the target tissue. In some aspects, the method further includes coupling a section of the proximally-extending portion of the elongated compressive element with respect to a second component of the delivery device to maintain the distal portion of the elongated compressive element in place surrounding the target tissue. In some aspects, the method further includes delivering the distalmost end of the elongated compressive element to the target tissue coupled to a first component of a cinch device. In some aspects, the method further includes delivering a second component of the cinch device with the proximally-extending portion of the elongated compressive element extending through an opening therethrough.

These and other features and advantages of the present disclosure, will be readily apparent from the following detailed description, the scope of the claimed invention being set out in the appended claims. While the following disclosure is presented in terms of aspects or embodiments, it should be appreciated that individual aspects can be claimed separately or in combination with aspects and features of that embodiment or any other embodiment.

### BRIEF DESCRIPTION OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. For example, devices (or even components thereof) may be enlarged so that detail is discernable, but may be intended to be scaled down in relation to a working channel of a delivery catheter or endoscope, e.g., to fit therein. In the figures, identical or nearly identical or equivalent elements are typically represented by the same reference characters. For purposes of clarity and simplicity, not every element is labeled in every figure, nor is every element of each embodiment shown where illustration is not necessary to allow those of ordinary skill in the art to understand the disclosure.

The detailed description will be better understood in conjunction with the accompanying drawings, wherein like reference characters represent like elements, as follows:

FIG. 1 illustrates a system for applying compressive pressure around tissue formed in accordance with aspects of the present disclosure.

FIG. 2 illustrates a compression device of a system as illustrated in FIG. 1 with a compressive element extending around target tissue.

FIG. 3 illustrates a compression device as in FIG. 2, with the compressive element in a further closed position around the target tissue.

FIG. 4 illustrates a compression device as in FIG. 2 and FIG. 3 in or approaching a

final state of compression by the compressive element.

FIG. 5 illustrates a compression as in FIGS. 2-4 being separated from the delivery device.

FIG. 6 illustrates an example of a tension adjuster by which tension on a compressive element as in FIGS. 2-5 may be adjusted.

FIG. 7 illustrates an example of an embodiment of a fitment which may be used to hold a compressive element as in FIGS. 2-5 in place to maintain compressive force on the target tissue.

### DETAILED DESCRIPTION

The following detailed description should be read with reference to the drawings, which depict illustrative embodiments. It is to be understood that the disclosure is not limited to the particular embodiments described, as such may vary. All apparatuses and systems and methods discussed herein are examples of apparatuses and/or systems and/or methods implemented in accordance with one or more principles of this disclosure. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. In fact, it will be apparent to those skilled in the art that various modifications and variations can be made in the present disclosure without departing from the scope or spirit of the present subject matter.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

As used herein, “proximal” refers to the direction or location closest to the user (medical professional or clinician or technician or operator or physician, etc., such terms being used interchangeably herein without intent to limit, and including automated controller systems or otherwise), etc., such as when using a device (e.g., introducing the device into a patient, or during implantation, positioning, or delivery), and/or closest to a delivery device, and “distal” refers to the direction or location furthest from the user, such as when using the device (e.g., introducing the device into a patient, or during implantation, positioning, or delivery), and/or closest to a delivery device. “Longitudinal” means extending along the longer or larger dimension of an element. A “longitudinal axis” extends along the longitudinal extent of an element, though is not necessarily straight and does not necessarily maintain a fixed configuration if the element flexes or bends, and “axial” generally refers to along the longitudinal axis. However, it will be appreciated that reference to axial or longitudinal movement with respect to the above-described systems or elements thereof need not be strictly limited to axial and/or longitudinal movements along a longitudinal axis or central axis of the referenced elements. “Central” means at least generally bisecting a center point and/or generally equidistant from a periphery or boundary, and a “central axis” means, with respect to an opening, a line that at least generally bisects a center point of the opening, extending longitudinally along the length of the opening when the opening comprises, for example, a tubular element, a strut, a channel, a cavity, or a bore. As used herein, a “lumen” or “channel” or “bore” or “passage” is not limited to a circular cross-section. As used herein, a “free end” of an element is a terminal end at which such element does not extend beyond. It will be appreciated that terms such as at or on or adjacent or along an end may be used interchangeably herein without intent to limit unless otherwise stated, and are intended to indicate a general relative spatial relation rather than a precisely limited location. Finally, reference to “at” a location or site is intended to include at and/or about the vicinity of (e.g., along, adjacent, proximate, etc.) such location or site, unless otherwise stated. As understood herein, corresponding is intended to convey a relationship between components, parts, elements, etc., configured to interact with or to have another intended relationship with one another.

Minimally-invasive procedures, which access a target site within the patient via a natural orifice (or, in some instances, a small incision not considered to constitute an open-surgery cut), are often preferable over open surgical procedure which require the patient to be cut open by a surgeon to access a target site within the patient. Such minimally invasive procedures include endoscopic, percutaneous, transcatheter, and/or transluminal procedures. Generally, when target tissue, within or in the area of the target site within a patient's body, is to be removed, or needs to be repaired, it is desirable to do so using a minimally-invasive procedure. It will be appreciated that terms such as target site, target area, target tissue site, target tissue area, target area of tissue, target treatment area, treatment area, target treatment site, treatment site, etc., may be used interchangeably herein, without intent to limit, to refer to an area or region of tissue within which a target tissue is located. The term target tissue (and other variations thereof, such as treatment tissue) is used herein to refer to the tissue with respect to which a procedure is to be performed or which is to be treated or otherwise operated on or affected by the devices and/or systems and/or methods disclosed herein. Thus, the target site may be understood as an area or region extending outwardly from or around or surrounding the target tissue (specific tissue in the target tissue area).

In accordance with various principles of the present disclosure, a system for applying compressive forces via a device having a component or element enclosing (or at least partially enclosing) target tissue is simplified over prior art systems. Various simplifications the present disclosure achieves over prior systems include, without limitation, fewer operative components to effect the application of force, and/or greater control (e.g., increasing or decreasing) of the force being applied, and/or greater control of the device applying the force. It will be appreciated that terms such as enclosing, surrounding, encircling, etc., may be used interchangeably herein to convey that an element extends around a perimeter of an area or object fully or at least partially. Compared to currently available ligation devices and systems, devices and systems of the present disclosure are configured to correct course in the sense that the compression device of the system includes a compressive element (e.g., a loop of an elongate flexible element) which can be adjusted, re-opened, etc., if needed, before full deployment of the device. In contrast, currently available devices typically cannot be adjusted, re-opened, etc., once the medical professional begins closing the device onto tissue (e.g., closing the loop).

In accordance with various principles of the present disclosure, the compression device of a system of the present disclosure includes a compressive element. In some aspects, the compressive element is formed of an elongate flexible element extendable into a patient from a proximal end remaining outside the patient (for control/manipulation by a medical professional). The compressive element has a distal portion configured to be positioned around (e.g., looped or otherwise formed to surround) target tissue within the patient, and a proximally-extending portion extending proximally from the distal portion and out of the patient's body toward the proximal end manipulated by a medical professional. Such configuration, with a proximal end of the compressive element available for control by the medical professional, while a distal portion is within the patient, allows the medical professional a better ability to judge the force applied to the distal portion of the compressive element to apply compressive forces to tissue. Alternatively or additionally, such configuration eliminates various components in prior art systems which transfer force applied by the medical professional to a proximal component (separately formed from the compressive element) distally to the compressive element at the distal end of the device which applies compressive force to the tissue.

In accordance with various principles of the present disclosure, the distal portion of the compressive element is sufficiently flexible to be extended in a generally closed configuration to enclose an area. For instance, the distal portion may be extended into a generally closed configuration around the perimeter of (e.g., to form a loop around) target tissue. In some aspects, the distal portion is configured to allow the medical professional to adjust the area enclosed by the distal portion. For the sake of convenience, and without intent to limit, the configuration of the distal portion to surround an area, such as to surround target tissue, may be referenced herein as a compression loop. In some aspects, the size of the area enclosed by the distal portion when formed into a compression loop may be increased and decreased without limit so that the medical professional may position, adjust, and optionally also readjust or reposition the loop. In some aspects, the distalmost end of the compressive element is maintained adjacent the target tissue, and the proximally-extending portion of the compressive element (extending proximally from the distal portion which is formed into the compression loop) is movable with respect to the distalmost end to adjust the area enclosed by the compression loop. The size of the compression loop may be decreased to contact the target tissue enclosed by the compression loop, and to apply a compressive force against the target tissue. In some aspects, the distalmost end of the compressive element is held in place with respect to target tissue by a component of a delivery device. In some aspects, a distal section of the proximally-extending portion of the compressive element and/or a proximal section of the distal portion (from which the proximally-extending portion extends) is held in place with respect to the distalmost end of the compressive element to form the generally closed configuration of the distal portion. In some aspects, the distal section of the proximally-extending portion and/or the proximal section of the distal portion is coupled with respect to the distalmost end via a component of the compression device and/or of the delivery device. In some aspects, the proximally-extending portion extends through an opening through such component to allow translational (e.g., axial, sliding) movement to adjust the configuration of the distal portion by retracting or extending the proximally-extending portion of the compressive element. It will be appreciated that terms such as opening, aperture, hole, passage, etc., may be used interchangeably herein without intent to limit.

In view of the limited working space within a patient (i.e., without using open surgery), the provision of a cinch device on (e.g., preloaded on) a compression device (such as formed in accordance with various principles of the present disclosure) facilitates completion of the procedure. More particularly, the incorporation of a cinch device into a compression device (e.g., by coupling a cinch element with respect to the compressive element for delivery therewith to a target site) facilitates deployment of the compression device and maintaining of the desired compressive force on the tissue encircled by the compressive element. In some aspects, the cinch device not only maintains the final desired configuration and applied force of the compressive element around the tissue, but also may be a part of the loop the compression device forms around the target tissue. More particularly, in some aspects, an element of the cinch device may define an aperture through which the proximally-extending portion of the compressive element (extending proximally from the distalmost, free end of the elongate flexible element) movably extends. With a free end of the compressive element maintained in place with respect to the target tissue, and a proximally-extending portion of the compressive element extending through a component of the cinch system, the cinch system facilitates the forming of the distal portion of the compressive element into a loop and/or facilitates adjustability of a loop of the compressive element with which compressive forces are to be applied to tissue. In some aspects, the distalmost, free end of the compressive element may be coupled to a component of the cinch device. In some aspects, movement of a proximally-extending portion of the compressive element through a component of the cinch device, and coupling of another component of the cinch device to the distalmost end of the compressive element provides a self-contained compression device with incorporated cinch device. Once the desired compressive force is achieved by the compression device, the incorporated cinch device may readily be actuated to hold the compressive element in place to maintain the desired compressive force which has been achieved.

In some aspects, a compression device formed in accordance with various principles of the present disclosure has a delivery configuration and a deployed configuration. In the delivery configuration, the compression device is configured to be delivered within the lumen of a tubular member of a delivery device of the system to a target site. In the deployed configuration, a distal portion of the compression device is extended outside and distal to the tubular member. In accordance with various principles of the present disclosure, at least one component of the cinch device is coupled to the distal portion of the compression device when the compression device is in the delivery configuration as well as in the deployed configuration.

Although the compressive element of a compression device and system formed in accordance with various principles of the present disclosure is generally described as being positioned around tissue, it will be appreciated that the configuration need not be circular, or even a traditional loop configuration (the term “loop” being used herein in the broadest sense for the sake of convenience and without intent to limit). Moreover, although applications of devices, systems, and methods formed in accordance with various principles of the present disclosure are described herein as used for bleeding control and prevention, ligation (e.g., of polyps, tumors, etc.), and/or closure of a tissue defect (e.g., perforations, wounds, cuts, such as, but not limited to those resulting from a medical procedure), the present disclosure is not limited in this regard and is applicable to various other procedures which may be contemplated by those of ordinary skill in the art.

Various embodiments of a system and devices for applying compressive forces around tissue, and associated methods, will now be described with reference to examples illustrated in the accompanying drawings. Reference in this specification to “one embodiment,” “an embodiment,” “some embodiments”, “other embodiments”, etc. indicates that one or more particular features, structures, concepts, and/or characteristics in accordance with principles of the present disclosure may be included in connection with the embodiment. However, such references do not necessarily mean that all embodiments include the particular features, structures, concepts, and/or characteristics, or that an embodiment includes all features, structures, concepts, and/or characteristics. Some embodiments may include one or more such features, structures, concepts, and/or characteristics, in various combinations thereof. It should be understood that any of the features, structures, concepts, and/or characteristics described herein can be mixed and matched to create hybrid embodiments, and such hybrid embodiment are within the scope of the present disclosure. It should further be understood that various features, structures, concepts, and/or characteristics of disclosed embodiments are independent of and separate from one another, and may be used or present individually or in various combinations with one another to create alternative embodiments which are considered part of the present disclosure. Therefore, the present disclosure is not limited to only the embodiments specifically described herein, as it would be too cumbersome to describe all of the numerous possible combinations and subcombinations of features, structures, concepts, and/or characteristics, and the examples of embodiments disclosed herein are not intended as limiting the broader aspects of the present disclosure. The following description is of illustrative examples of embodiments only, and is not intended as limiting the broader aspects of the present disclosure.

In the following description and accompanying drawings, it will be appreciated that common features are identified by common reference elements and, for the sake of brevity and convenience, and without intent to limit, the descriptions of the common features are generally not repeated. For purposes of clarity, not all components having the same reference number are numbered.

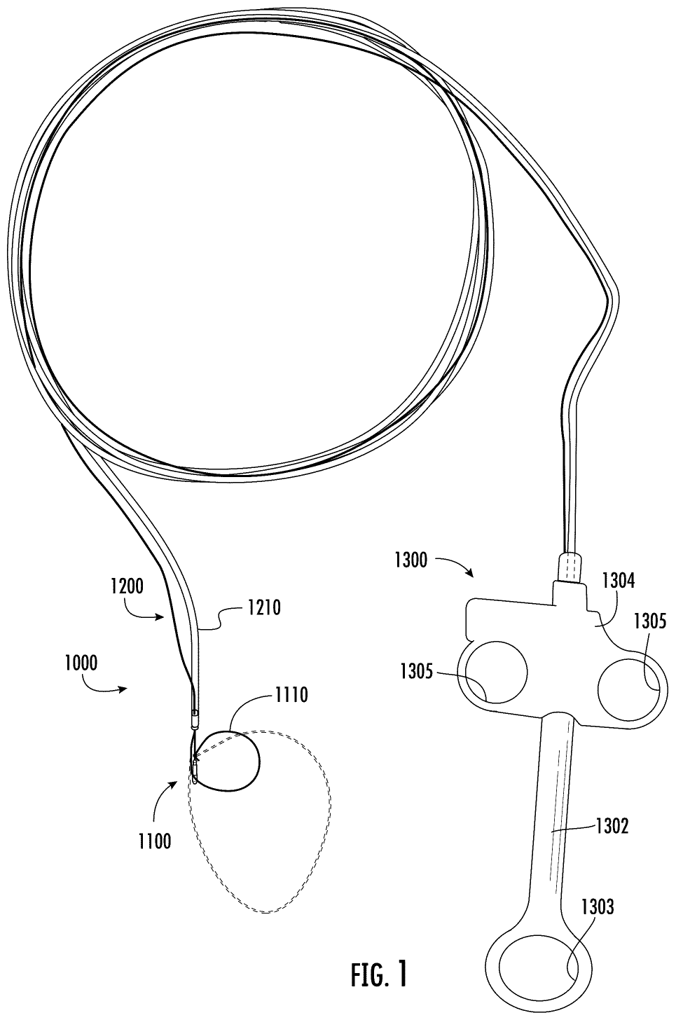

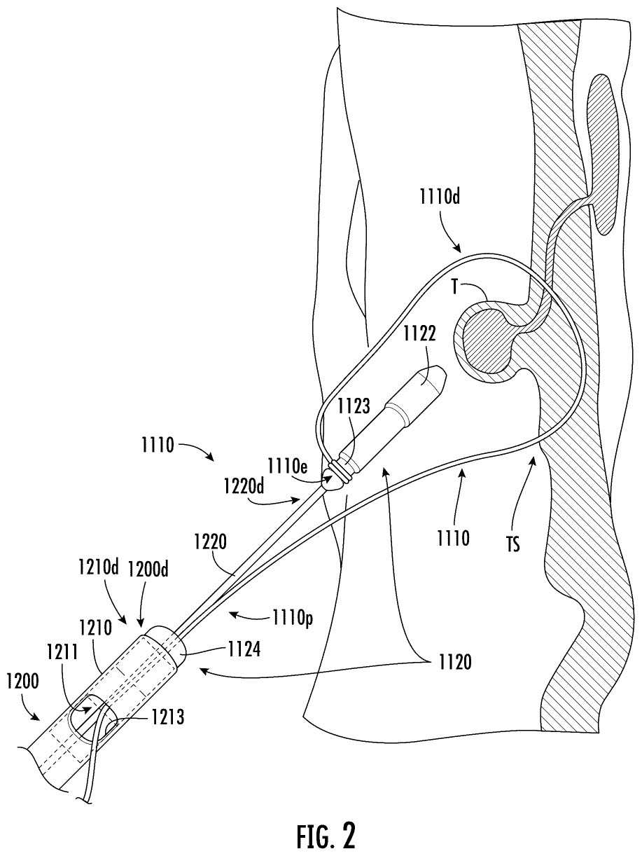

Turning now to the drawings, an example of an embodiment of a system 1000 for applying compressive pressure around tissue (e.g., radially inwardly circumferential pressure) is illustrated in FIG. 1. The system 1000 includes a compression device 1100 having a compressive element 1110 configured to surround tissue to apply compressive force around the tissue, and a delivery device 1200 configured to deliver the compression device 1100 to a target site within a patient. More particularly, in the illustrated example of an embodiment, the compressive element 1110 is configured to ligate or otherwise to apply compressive forces to tissue, such as, without limitation, to ligate and remove (and/or to cause necrosis and subsequent separation from the tissue surrounding) the target tissue, to repair an opening in the target tissue, to control or prevent bleeding, etc. In some aspects, the compressive element 1110 surrounds, encircles, loops around, etc. (such terms being used interchangeably herein without intent to limit), or otherwise is configured to be placed around the perimeter of target tissue at the target site. In some aspects, the compressive element 1110 is configured to apply a radially-inwardly directed force to the target tissue, as described in further detail below. In some aspects, the delivery device 1200 includes a tubular member 1210 defining a longitudinal lumen 1211 therethrough (see, e.g., FIG. 2). The compressive element 1110 (or at least a portion thereof) may be delivered to the target site within and through the lumen 1211 of the tubular member 1210, or within only a distal portion of the lumen 1211 of the tubular member 1210, with the proximal portion of the compressive element 1110p extending along the exterior of the tubular member 1210 (such as illustrated in FIG. 1). In some aspects, a connector 1202 is operatively associated with the delivery device 1200 and configured to maintain the proximal portion of the compressive element 1110p adjacent the connector 1202. In some aspects, the target site is within a patient's body, and the tubular member 1210 is sized (e.g., elongated and/or having a sufficiently small outer diameter) and sufficiently flexible to be inserted into a natural orifice of (or small incision in) the patient's body to navigate within the patient's body, such as through tortuous passages therein, to a target site. However, it will be appreciated that in some procedures, a more rigid tubular delivery element may be desired.

The compressive element 1110 is arranged and configured to form an enclosed distal portion 1110d to extend around target tissue T, such as illustrated in FIGS. 2-5. In the example of an embodiment illustrated in FIG. 2, the compressive element 1110 is illustrated as an elongate element, such as, without limitation, a filament, string, suture, wire, cord, dental floss, etc., such terms being used interchangeably herein without intent to limit. The compressive element 1110 has a flexible distal portion 1110d capable of extending around target tissue T, such as by being sufficiently flexible to extend around the target tissue T, such as in the form of a loop or lasso. It will be appreciated that terms such as loop, lasso, etc., are used interchangeably herein to describe a portion of the compressive element 1110 enclosing (fully or just partially) an area, such as to surround tissue. The compressive element 1110 may be inelastic or elastic, generally non-stretchable or stretchable, generally limp (e.g., not maintaining a shape on its own) or sufficiently stiff to maintain a shape independently of another element, and/or may have other characteristics suitable for extending around tissue and applying radially-inward compressive force to the surrounded tissue, as may be appreciated by those of ordinary skill in the art.

In accordance with various principles of the present disclosure, the compressive element 1110 is movable within the patient's body to adjust the size of the loop formed by the distal portion 1110d thereof, such as to increase compressive force applied by the distal portion 1110d to the tissue surrounded by the distal portion 1110d, such as target tissue T. In some aspects, the proximally-extending portion 1110p of the compressive element 1110 extends proximally from a proximal section of the looped distal portion 1110d of the compressive element 1110 and is movable with respect to the target tissue T to increase or decrease the size of the distal portion 1110d surrounding the target tissue T. In some aspects, the proximally-extending portion 1110p of the compressive element 1110 extends proximally to a position outside the patient's body to the proximal end of the compressive element 1110 to allow a medical professional direct access to the compressive element 1110 to adjust the looped distal portion 1110d of the compressive element 1110 by extending (e.g., pushing, distally advancing, etc.) or retracting (e.g., pulling, proximally withdrawing, etc.) the compressive element 1110 with respect to the patient's body.

To facilitate adjustment of the looped distal portion 1110d of the compressive element 1110, the distalmost end 1110e of the compressive element 1110 is positioned and maintained at the target site TS while the proximally-extending portion 1110p of the compressive element 1110 is movable with respect to the target site TS. In some aspects, such as illustrated the example of an embodiment illustrated in FIGS. 2-5, a first component of the delivery device 1200 maintains the distalmost end 1110e of the compressive element 1110 in position with respect to the target site TS. In some aspects, the distalmost end 1110e of the compressive element 1110 is fixedly coupled to such first component of the delivery device 1200 to move therewith, and/or not to move with respect thereto. In some aspects, the delivery device 1200 includes an elongate member 1220 (e.g., a shaft, stylet, pull wire, etc.) movable with respect to the tubular member 1210 of the delivery device 1200 and with respect to which the distalmost end 1110e of the compressive element 1110 is fixedly coupled. The tubular member 1210 may thus be moved towards the target tissue T to position the distalmost end 1110e of the compressive element 1110 adjacent or in contact with the target tissue T. In some aspects, the elongate member 1220 extends through the lumen 1211 defined within the tubular member 1210, and is longitudinally translatable therein and therethrough. In some aspects, the elongate member 1220 is distally advanceable from the distal end 1200d of the delivery device 1200, such as out of a distal opening defined in the distal end 1210d of the tubular member 1210, to advance the distalmost end 1110e of the compressive element 1110 to the target site TS.

As noted above, the proximally-extending portion 1110p of the compressive element 1110 is movable with respect to the distalmost end 1110e of the compressive element 1110 to adjust the looped distal portion 1110d of the compressive element 1110 while the distalmost end 1110e is positioned and maintained at the target site TS. In some aspects, the proximally-extending portion 1110p of the compressive element 1110 is coupled with respect to a second component of the delivery device 1200 with the distalmost end 1110e coupled to a first component of the delivery device 1200. The relative positions of the first and second components of the delivery device 1200 may be maintained to maintain the distal portion 1110d of the compressive element 1110 in a configuration surrounding target tissue T. In some aspects, the proximally-extending portion 1110p of the compressive element 1110 may be coupled with respect to the delivery device 1200 to be movable with respect to the elongate member 1220 of the delivery device 1200 and thus with respect to the distalmost end 1110e of the compressive element 1110. The first component and the second component of the delivery device 1200 may be movable with respect to each other so that the proximally-extending portion 1110p of the compressive element 1110 (coupled to the second component) may be moved relative to the distalmost end 1110e of the compressive element 1110 (coupled to the first component) to adjust the looped distal portion 1110d of the compressive element 1110. In some aspects, the proximally-extending portion 1110p of the compressive element 1110 is movably coupled with respect to the second component of the delivery device 1200 to move with respect to the first component of the delivery device and thus with respect to the distalmost end 1110e of the compressive element 1110 coupled thereto. In such configuration, the first component and the second component of the delivery device 1200 may or may not be moved with respect to each other to move the proximally-extending portion 1110p of the compressive element 1110. In some aspects, the proximally-extending portion 1110p of the compressive element 1110 is movable through a lumen or opening within or defined by the second component of the delivery device 1200. In some aspects, the second component is the tubular member 1210 of the delivery device 1200, and the proximally-extending portion 1110p of the compressive element 1110 is movable within the lumen 1211 defined therein. In some aspects, the proximally-extending portion 1110p of the compressive element 1110 extends into the opening defined in the distal end 1210d of the tubular member 1210 and proximally through the lumen 1211 therein for grasping and manipulation by a medical professional. It will be appreciated that the proximally-extending portion 1110p of the compressive element 1110 may alternatively extend along the exterior of the tubular member 1210 and/or within another lumen defined in the tubular member 1210 or another member of the system 1000. For instance, the proximally-extending portion 1110p of the compressive element 1110 may initially extend into the opening defined in the distal end 1210d of the tubular member 1210, and then extend transversely (with respect to a longitudinal axis of the tubular member 1210) out of an opening 1213 defined in a wall of the tubular member 1210. Such arrangement may provide certain benefits, as described in further detail below.

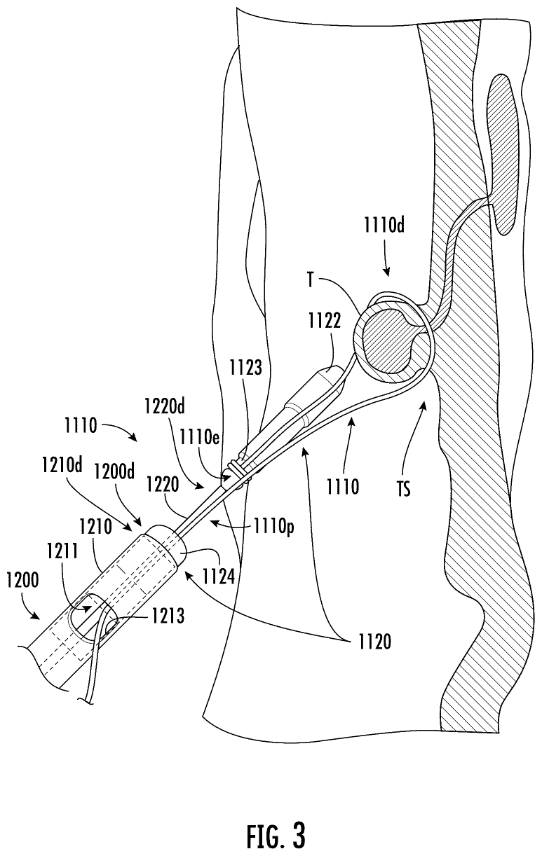

The proximally-extending portion 1110p of the compressive element 1110 extends proximally to be manipulated by a medical professional to modify the size of the area enclosed by the distal portion 1110d to surround the target tissue T as desired and to apply compressive force (e.g., radially-inward force) to the target tissue T. It will be appreciated that terms such as manipulate, move, control, adjust, etc., and other grammatical forms thereof, may be used interchangeably herein without intent to limit unless otherwise stated. Proximal movement of the proximally-extending portion 1110p of the compressive element 1110 relative to the distalmost end 1110e reduces the size of the looped distal portion 1110d, such as illustrated in FIG. 3 (e.g., as compared with the configuration of the looped distal portion 1110d illustrated in FIG. 2). In some aspects, the proximally-extending portion 1110p of the compressive element 1110 is freely movable (e.g., proximally or distally) with respect to the distalmost end 1110e of the compressive element 1110. As such, the proximally-extending portion 1110p of the compressive element 1110 may be moved (e.g., distally) to increase the size of the looped distal portion 1110d with respect to the target tissue T.

In view of the above, it will be appreciated that a simple loop or lasso is formed with a flexible distal portion 1110d of the compressive element 1110 which is adjustable to surround the target tissue T (e.g., the size of the loop or lasso may be increased or decreased as needed to surround the target tissue T), as well as to apply or reduce compressive force to the target tissue T. For instance, if the target tissue T is pedunculated, with a free end larger than a peduncle coupling the free end to the target site TS, then it may be necessary to increase the size of the loop formed by the distal portion 1110d of the compressive element 1110 to pass over the enlarged free end to surround the peduncle. The medical professional may grasp the proximal end of the compressive element 1110 either directly, or via another grasping element, to adjust the compression on the target tissue T applied/created by the compressive element 1110. Additional elements, such as in prior systems, coupling the member which is controlled by the medical professional and the member which surround the target tissue T to apply compressive force thereto are not needed. As may be appreciated, the proximal end of the compressive element 1110 is manipulated by the medical professional, and the distalmost end 1110e of the same compressive element 1110 is positioned at the target tissue T, with a distal portion 1110d of the same compressive element 1110 surrounding target tissue T. Moreover, a greater degree of adjustability is provided by the present disclosure compared to various prior systems. For instance, two-way adjustability of the looped distal portion 1110d of the compressive element 1110 of the compression device 1100 allows tightening or loosening of the distal portion 1110d around the target tissue T.

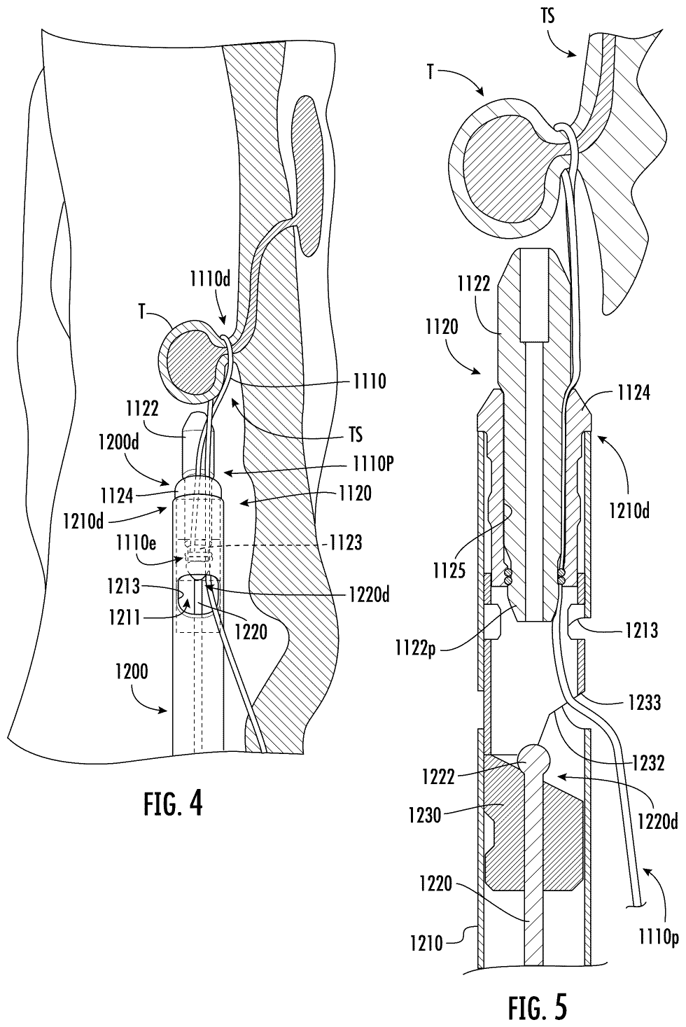

As may be appreciated with reference to FIG. 4, continued proximal movement of the proximally-extending portion 1110p of the compressive element 1110 with respect to the distalmost end 1110e continues to reduce the size of the looped distal portion 1110d until the compressive element 1110 contacts the periphery of the target tissue T. The medical professional may continue to proximally retract the proximally-extending portion 1110p of the compressive element 1110 to apply compressive force to the surrounded target tissue T until the desired effect is reached. As may be appreciated, once the desired compressive force of the compressive element 1110 with respect to the target tissue T is attained, it is desirable to restrain or fix relative movement of the proximally-extending portion 1110p of the compressive element 1110 with respect to the distalmost end 1110e. Various manners of maintaining the desired configuration of the distal portion 1110d of the compressive element 1110 may be used, such as formation of a knot, or application of a separate element with respect to the compressive element 1110 to hold the relative position of the distalmost end 1110e with respect to the proximally-extending portion 1110p of the compressive element 1110.

In accordance with various principles of the present disclosure, the compression device 1100 may further include a cinch device 1120 operatively coupled with respect to and delivered with the compressive element 1110 to the target site TS. The cinch device 1120 is actuatable between an unlocked configuration allowing adjustability of the distal portion 1110d of the compressive element 1110, and a locked configuration maintaining the distal portion 1110d in a configuration which applies the desired amount of compression on the target tissue T. In some aspects, the cinch device 1120 is operatively coupled with respect to the delivery device 1200 in addition to being operatively coupled with the compressive element 1110. In some aspects, the cinch device 1120 includes a first component and a second component movable with respect to each other to shift the cinch device 1120 between the unlocked configuration and the locked configuration. Once a desired size of the distal portion 1110d of the compressive element 1110 surrounding the target tissue T and/or the desired compression of the target tissue T via the compressive element 1110 has been reached/attained, the cinch device 1120 is actuated or otherwise moved from the unlocked configuration (e.g., such as illustrated in FIG. 2 and FIG. 3) to a locked configuration (e.g., such as illustrated in FIG. 5). The position of the distalmost end 1110e may thereby be fixed with respect to the proximally-extending portion 1110p of the compressive element 1110, and thereby maintain the configuration of the distal portion 1110d and the compression it applies to the target tissue T. In some aspects, the first component of the cinch device 1120 is coupled with respect to the distalmost end 1110e of the compressive element 1110, and is movable with respect to the second component of the cinch device 1120. In some aspects, at least one of the first component or the second component of the cinch device 1120 is operatively coupled with a component movable by the medical professional. In some aspects, at least one of the first component or the second component of the cinch device 1120 is operatively coupled with a component of the delivery device 1200. In some aspects, the first and second components of the cinch device 1120 are coupled to different components of the delivery device 1200 which are movable with respect to each other so that the first and second components of the cinch device 1120 are movable with respect to each other to shift the cinch device 1120 between the unlocked and locked configurations. In embodiments in which the first component of the cinch device 1120 is operatively coupled with respect to the distalmost end 1110e of the compressive element 1110, the first component of the cinch device 1120 may also be coupled to the elongate member 1220 of the delivery device 1200. In some aspects, the distalmost end 1110e of the compressive element 1110 may be fixed, via the first component of the cinch device 1120, with respect to the elongate member 1220. Movement of the elongate member 1220 thus moves the first component of the cinch device 1120 together with the distalmost end 1110e end of the compressive element 1110.

In some aspects, the first component of the cinch device 1120 is coupled to the elongate member 1220 of the delivery device 1200 (e.g., to the distal end 1220d of the elongate member 1220, such as illustrated in FIG. 2), and the second component of the cinch device 1120 is coupled to the tubular member 1210 through which the elongate member 1220 extends. In such embodiment, relative movement of the elongate member 1220 and the tubular member 1210 of the delivery device 1200 causes relative movement of the first component and the second component of the cinch device 1120 to shift the cinch device 1120 between the unlocked and locked configurations. For instance, the first component of the cinch device 1120 may be movable with respect to the second component of the cinch device 1120 by longitudinal translation of the elongate member 1220 within the lumen 1211 of the tubular member 1210 and/or longitudinal translation of (advancing or retracting) the tubular member 1210 over the elongate member 1220. Alternatively or additionally, the first component of the cinch device 1120 may be moved (such as upon movement of the elongate member 1220) into a lumen formed in the second component of the cinch device 1120 (e.g., through which the elongate member 1220 extends as well) to shift the cinch device 1120 into a locked configuration. It will be appreciated that the second component of the cinch device 1120 need not be coupled with the tubular member 1210 or any other member or component of the delivery device 1200.

It will be appreciated that the manner in which the cinch device 1120 is incorporated into the system 1000 does not complicate or otherwise impact the above-described simple, streamlined design of the compressive element 1110 of the present disclosure. For instance, operative coupling of the first and second components of the cinch device 1120 with other components of the system 1000 (e.g., with the compressive element 1110 and/or one or more components of the delivery device 1200) simplifies construction and use of the system 1000. For instance, the first component of the cinch device 1120 may be operatively coupled with the compressive element 1110 to move therewith. As such, the control element used to cause movement of the distalmost end 1110e of the compressive element 1110 may also be used to cause movement of the first component of the cinch device 1120. Likewise, the same element which holds the distalmost end 1110e of the compressive element 1110 with respect to target tissue T may be used to hold the first component of the cinch device 1120 in place while a second component of the cinch device 1120 is moved thereto to shift the cinch device 1120 into a locked configuration. For instance, in the example of an embodiment illustrated in FIGS. 2-5, the elongate member 1220 of the delivery device 1200 may be used to advance the distalmost end 1110e of the compressive element 1110 to target tissue T, and to maintain the position of the distalmost end 1110e with respect to the target tissue T, as well as to move the cinch element 1122 and to hold the cinch element 1122 in place while the cinch collar 1124 is moved into engagement with the cinch element 1122. Moreover, operative coupling of the second component of the cinch device 1120 with the delivery device 1200 allows manipulation, adjustment, movement, control, etc., of the second component via the delivery device 1200, without requiring further control elements. For instance, in the example of an embodiment illustrated in FIGS. 2-5, the compression device 1100 is delivered within the tubular member 1210 of the delivery device 1200, and the cinch collar 1124 is also delivered to the target site TS with the tubular member 1210 as well as moved by the tubular member 1210 into engagement with the cinch element 1122.

An example of an embodiment of a system 1000 for applying compressive pressure around tissue in accordance with various principles of the present disclosure, and which includes a cinch device 1120 incorporated into the system 1000, is illustrated in FIGS. 2-5. The illustrated system 1000 includes a delivery device 1200, a compressive element 1110, and a cinch device 1120 having a first component in the form of a cinch element 1122 and a second component in the form of a cinch collar 1124. In the illustrated example of an embodiment of a cinch device 1120, the cinch element 1122 and the distalmost end 1110e of the compressive element 1110 are fixedly coupled together. For instance, the distalmost end 1110e of the compressive element 1110 may be wrapped around, looped and optionally also knotted, adhered, welded, or otherwise coupled to the cinch element 1122. In some aspects, the cinch element 1122 includes a coupling groove 1123 with respect to which (e.g., about which) the distalmost end 1110e of the compressive element 1110 is positioned (and fixed). The elongate member 1220 of the delivery device 1200 is coupled to the cinch element 1122 (e.g., an end, or other portion, thereof) to thereby move the distalmost end 1110e of the compressive element 1110 with respect to the target site TS. It will be appreciated that the cinch element 1122 and the distalmost end 1110e of the compressive element 1110 may be directly coupled together to simplify the system 1000. The illustrated example of an embodiment of a cinch device 1120 further includes a cinch collar 1124 operatively coupled with a distal end 1210d of the tubular member 1210 of the delivery device 1200.

It will be appreciated that, as noted above, it may be desirable for the proximally-extending portion 1110p of the compressive element 1110 to be associated with the distalmost end 1110e (e.g., spatially, such as in sufficiently close proximity) to form a shape enclosing target tissue T. To allow relative movement of the proximally-extending portion 1110p with respect to the distalmost end 1110e of the compressive element 1110, the proximally-extending portion 1110p may be coupled/associated with the distalmost end 1110e of the compressive element 1110 via an opening in a component of the system 1000. In the example of an embodiment illustrated in FIGS. 2-5, and as noted above, the proximally-extending portion 1110p of the compressive element 1110 may extend into the opening at the distal end 1210d of the tubular member 1210 and then laterally out of the tubular member 1210 via an opening 1213 defined in the wall of the tubular member 1210. The section of the proximally-extending portion 1110p of the compressive element 1110 which extends into the lumen 1211 of the tubular member 1210 is thereby maintained adjacent the portion of the elongate member 1220 within the lumen 1211. Since the distalmost end 1110e of the compressive element 1110 is coupled to the elongate member 1220 (which extends through the tubular member 1210), positioning of a section of the proximally-extending portion 1110p of the compressive element 1110 adjacent the tubular member 1210 positions such section of the proximally-extending portion 1110p of the compressive element 1110 in sufficient proximity to the distalmost end 1110e of the compressive element 1110 to form the distal portion 1110d of the compressive element 1110 into a generally enclosed shape. The medical professional may thus manipulate the proximal end of the compressive element 1110 with respect to the tubular member 1210, without disturbing the configuration of the distal portion 1110d as surrounding the target tissue T. For instance, the medical professional may hold the delivery device 1200 in place once the distalmost end 1110e of the compressive element 1110 and/or the cinch element 1122 have been positioned adjacent (e.g., immediately adjacent, such as in contact with) the target tissue T. The medical professional may pull proximally on the compressive element 1110 to tighten the distal portion 1110d thereof around the target tissue T while holding the distalmost end 1110e of the compressive element 1110 (along with the cinch element 1122 coupled thereto) close to the target tissue T, such as illustrated in FIG. 3 and FIG. 4. The delivery device 1200 may be proximally withdrawn from the compression device 1100 to deploy the compression device 1100 with respect to the target tissue T. For instance, the elongate member 1220 may be decoupled from the distalmost end 1110e of the compressive element 1110, such as by being decoupled from the cinch element 1122, and the tubular member 1210 may be decoupled from the cinch collar 1124.

Another advantage of extending the proximally-extending portion 1110p of the compressive element 1110 laterally out of the lumen 1211 of the tubular member 1210 via the opening 1213, as described above, is the resulting placement of the proximally-extending portion 1110p to be separated from the distal portion 1110d of the compressive element 1110 looped around the target tissue T. For instance, in the example of an embodiment illustrated in FIG. 5, the delivery device 1200 includes a cutter 1230 positioned within the tubular member 1210 and movable by the elongate member 1220. The cutter 1230 defines a lateral opening 1233 which may be generally aligned with the opening 1213 in the wall of the tubular member 1210 while the distal portion 1110d of the compressive element 1110 is being positioned and adjusted to apply compressive force to the target tissue T. Thus, the proximally-extending portion 1110p of the compressive element 1110 extends through the lateral opening 1233 in the cutter 1230 as well as the opening 1213 in the wall of the tubular member 1210. To deploy the cinch device 1120 (in a locked configuration) and the compressive element 1110, the elongate member 1220 is withdrawn proximally to separate from the cinch element 1122. The elongate member 1220 may have an enlarged distal end 1222 which releasably couples the elongate member 1220 with the cinch element 1122 (e.g., by an interference or friction fit). Application of sufficient proximal force to the elongate member 1220 overcomes the friction fit of the enlarged distal end 1222 with respect to the cinch element 1122 and withdraws the elongate member 1220 from the cinch element 1122. The cutter 1230 includes a distal cutting edge 1232 along a distal edge of the lateral opening 1233. Proximal withdrawal of the elongate member 1220 engages the enlarged distal end 1222 with the cutter 1230 and moves the cutter 1230 proximally to move the cutting edge 1232 of the cutter 1230 into engagement with a portion of the compressive element 1110. Continued proximal withdrawal of the elongate member 1220 and the cutter 1230 causes the cutting edge 1233 of the cutter 1230 to sever the compressive element 1110. Continued proximal withdrawal of the elongate member 1220 may also proximally withdraw and release the tubular member 1210 from a friction fit or interference fit with respect to the cinch collar 1124, such as by engaging (or pulling the cutter 1230 into engagement with) an inwardly-extending projection within the lumen 1211 of the tubular member 1210. Various arrangements for disengaging the delivery device 1200 from the compression device 1100 may be used from various known systems, such as, without limitation, the systems described in any of U.S. Patent Application Publication 2021/0128140 to Wales et al., which published on May 6, 2021; U.S. Pat. No. 11,553,909 to Gilbert et al., which issued on Jan. 17, 2023; U.S. Pat. No. 8,540,735 to Mitelberg et al., which issued on Sep. 24, 2013. The cinch device 1120 is thereby deployed on the compressive element 1110, holding the compressive element 1110 in the desired configuration with respect to target tissue T, applying the desired amount of compressive force to the target tissue T.

In some aspects, a simple handle 1300, such as illustrated in FIG. 1, may be provided to move the elongate member 1220 with respect to the tubular member 1210. For instance, a common configuration such as known to those of ordinary skill in the art may include a first handle component 1302 fixed with respect to the tubular member 1210 (through which elongate member 1220 extends), and a second handle component 1304 fixed with respect to the elongate member 1220 and movable with respect to the first handle component 1302. A thumb ring/grip 1303 and finger grips 1305 may be respectively formed in the first handle component 1302 and the second handle component 1304 to facilitate grasping and manipulation thereof. Such handle 1300 may be considered a component of the delivery device 1200.

In accordance with various principles of the present disclosure, various additional components may be added to the above-described system 1000 for applying compressive pressure around tissue. For instance, components may be added to facilitate maintaining a desired withdrawal force on the compressive element 1110 prior to actuation and locking of the cinch device 1120 to hold the proximally-extending portion 1110p of the compressive element 1110 fixed in place with respect to the distalmost end 1110e and at a desired compressive configuration around the tissue.

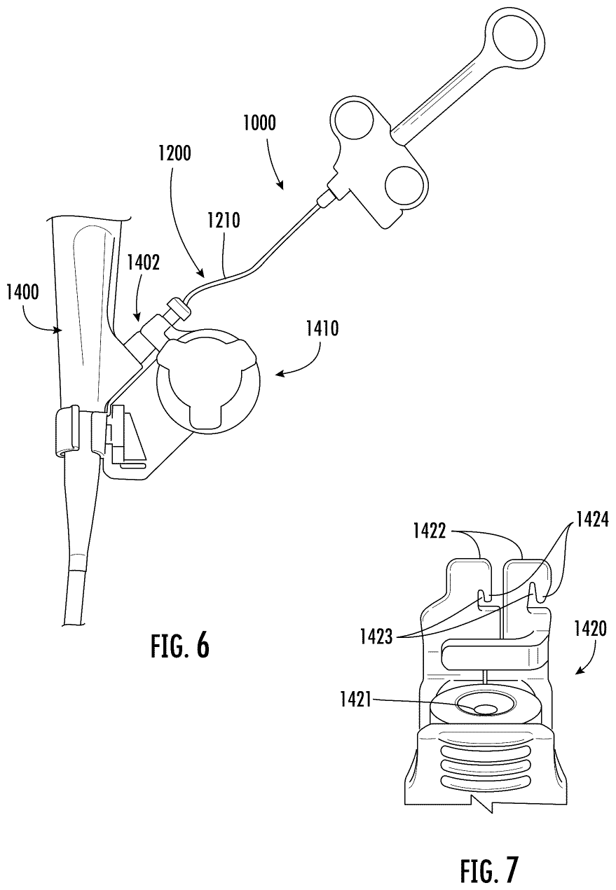

In the example of an embodiment of a system 1000 illustrated in FIG. 6, the system 1000 is used with an endoscope 1400 (which may be considered to be a component of the system 1000), with the delivery device 1200 (e.g., the tubular member 1210 thereof) extending through a side port 1402 of the endoscope 1400. A tension adjuster 1410, such as in the form of a rotatable knob, may be mounted on the endoscope 1400, such as on the side port 1402 as well. A proximal portion of the compressive element 1110 may extend proximally along the delivery device 1200, out the side port 1402, and into operable engagement with the tension adjuster 1410. Movement, such as rotation, of the tension adjuster 1410 causes distal advancement or proximal retraction of the compressive element 1110 with respect to the delivery device 1200 to adjust the distal portion 1110d of the compressive element 1110 in a manner such as described above. In some aspects, the tension adjuster 1410 may provide audible or tactile or visual feedback to the medical professional to indicate the amount (e.g., the length) the compressive element 1110 has been advanced/extended or withdrawn/retracted, and/or the amount of tension applied by the compressive element 1110, and/or other parameters with respect to the compressive element 1110 which are relevant to the procedure being performed with respect to the target tissue T. It will be appreciated that instead of an endoscope as illustrated in FIG. 6, another outer tubular delivery element (e.g., catheter, sheath, etc.) may be provided, with the delivery device 1200 and compressive element 1110 of the compression device 1100 extending through a lumen of such outer tubular delivery element to be delivered and deployed at a target site TS, and with the tension adjuster 1410 coupled to an external feature of such outer tubular delivery element.

If the medical professional prefers a simpler system 1000 and/or direct access to the proximal end of the compressive element 1110 of a system 1000 as described with respect to FIGS. 1-5, a fitment 1420 such as illustrated in FIG. 7 may be used to hold the desired force on the compressive element 1110 while the cinch device 1120 is being deployed. More particularly, a fitment 1420 such as illustrated in FIG. 7 may be configured to be inserted over a side port 1402 of an endoscope 1400 such as illustrated in FIG. 6, or over a component of another outer tubular delivery element through which the delivery device 1200 is delivered. The proximal end of the compressive element 1110 may thus extend out of the endoscope or outer tubular delivery element and through an opening 1421 in the fitment 1420 (which may be generally aligned with an opening in the side port 1402 with which the fitment 1420 is coupled) to be grasped and manipulated by a medical professional. When the desired configuration of the distal portion 1110d of the compressive element 1110 has been achieved with respect to the target tissue T, and/or the desired compressive force has been attained by the looped distal portion 1110d, the compressive element 1110 may be engaged with a gripper 1422 of the fitment 1420. More particularly, the grippers 1422 include a locking groove 1423 in which the compressive element 1110 may be extended and locked, and/or about which the compressive element 1110 may be wrapped or otherwise engaged, with an optional hook portion 1424 maintaining/holding the proximal end of the compressive element 1110 in place with respect to the gripper 1422 and thus with respect to the delivery device 1200. The cinch device 1120 may thus be actuated and deployed while the compressive element 1110 maintains the desired compressive force on target tissue T.

It will be appreciated that the distalmost end 1100e of the compressive element 1110 and the cinch element 1122 are illustrated spaced apart from the target tissue T for the sake of clarity to allow the features of the cinch element 1122 to be clearly illustrated but not necessarily to accurately represent the placement of cinch element 1122 when the medical professional begins to tighten the distal portion 1110d of the compressive element 1110 around the target tissue T in a manner as described above. Moreover, it will be appreciated that the relative proportions of the various components of the system 1000 and the target tissue T may not be accurate, and the components and target tissue T may not be illustrated along the same size scale.

It will be appreciated that although various components of the cinch device 1120 described herein may be familiar to those of ordinary skill in the art, such components have been inserted over a suture used to form suture stitches in tissue, or an element which is otherwise passed through tissue. Therefore, prior similar cinch devices have not shown or suggested coupling of any portion (distalmost end or otherwise) of an elongate flexible element which engages tissue, such as a suture, to the cinch element of the cinch device. Instead, the proximal end of the cinch element is coupled to a shaft which manipulates the position of the cinch element with respect to a cinch collar through which the tissue-engaging elongate flexible element (e.g., suture) extends. The distalmost end of the suture in prior cinch devices remains coupled to the target tissue, with only a portion of the suture extending proximally from the tissue and through the cinch collar. In contrast, the cinch device 1120 used with a system 1000 for applying compressive pressure around tissue as described herein includes a cinch element 1122 coupled to a compressive element 1110 (such as in the form of an elongate flexible element or otherwise) which engages tissue. The cinch element 1122 is thus delivered and deployed with the compressive element 1110 to a position adjacent target tissue T (but is not extended through tissue, such as with a suture extended through tissue). Moreover, whereas the prior cinch devices are applied after completing placement of a tissue-engaging elongate flexible element with respect to tissue (e.g., after finishing stitching tissue with the suture), the cinch device 1120 of the presently disclosed system 1000 is deployed with the compression device 1100 and coupled thereto while the compression device 1100 is positioned with respect to tissue, and manipulated with respect to tissue and into its final configuration. In some aspects, the proximally-extending portion 1110p of the compressive element 1110 extends through a component (e.g., the cinch collar 1124) of the cinch device 1120 while being manipulated to achieve the desired compressive force to be applied by the compression device 1100.

The foregoing discussion has broad application and has been presented for purposes of illustration and description and is not intended to limit the disclosure to the form or forms disclosed herein. It is to be understood by one of ordinary skill in the art that the present discussion is a description of illustrative examples of embodiments only, and is not intended as limiting the broader aspects of the present disclosure. The system and devices discussed herein are examples implemented in accordance with one or more principles of this disclosure. These examples are not the only way to implement these principles but are merely examples, not intended as limiting the broader aspects of the present disclosure. Thus, references to elements or structures or features in the drawings must be appreciated as references to examples of embodiments of the disclosure, and should not be understood as limiting the disclosure to the specific elements, structures, or features illustrated. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, such as which may be adapted to specific environments and operative requirements, without departing from the concept, spirit, or scope, or characteristics thereof. For example, elements shown as integrally formed may be constructed of multiple parts or elements shown as multiple parts may be integrally formed, the operation of elements may be reversed or otherwise varied, the size or dimensions of the elements may be varied. Other examples of manners of implementing the disclosed principles will occur to a person of ordinary skill in the art upon reading this disclosure. It should be apparent to those of ordinary skill in the art that variations can be applied to the disclosed devices, systems, and/or methods, and/or to the sequence of steps of the method described herein without departing from the concept, spirit, and scope of the disclosure. While the disclosure is presented in terms of embodiments, it should be appreciated that the various separate features of the present subject matter need not all be present in order to achieve at least some of the desired characteristics and/or benefits of the present subject matter or such individual features. Additionally, other implementations are within the scope of the following claims. Various further benefits of the various aspects, features, components, and structures of a system for applying compressive forces around tissue, and associated devices and methods, such as described above, in addition to those discussed above, may be appreciated by those of ordinary skill in the art.

It should be appreciated that the presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. As used in this specification and the appended claims, the term “or” is generally employed in its sense including “and/or” unless the content clearly dictates otherwise. As used herein, the conjunction “and” includes each of the structures, components, features, or the like, which are so conjoined, unless the context clearly indicates otherwise, and the conjunction “or” includes one or the others of the structures, components, features, or the like, which are so conjoined, singly and in any combination and number, unless the context clearly indicates otherwise. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, engaged, joined, etc.) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The following claims are hereby incorporated into this Detailed Description by this reference, with each claim standing on its own as a separate embodiment of the present disclosure. In the claims, the terms “comprises”, “comprising”, “includes”, and “including” do not exclude the presence of other elements, components, features, groups, regions, integers, steps, operations, etc. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. A system for applying compressive forces around target tissue, said system comprising:
a delivery device; and
a compression device comprising a compressive element having a flexible distal portion configured to surround the target tissue and ending in a distalmost end, and a proximally-extending portion extending proximally from the distal portion to a proximal end positionable outside the patient's body to be movable by a medical professional with respect thereto to adjust the size of the distal portion surrounding the target tissue to adjust compressive force on the target tissue.

2. The system of claim 1, further comprising a cinch device having a first component coupled to said compressive element for delivery therewith by said delivery device to the target tissue.

3. The system of claim 2, wherein said cinch device further comprises a second component coupled to said delivery device for delivery therewith to the target tissue, said first component and said second component movable between an unlocked configuration in which said first component and said second component are spaced apart from each other, and a locked configuration in which said first component and said second component are coupled together.

4. The system of claim 3, wherein:
said first component of said cinch device is coupled to a distalmost end of said compressive element and to a first component of said delivery device movable with respect to a second component of said delivery device to position said compressive element with respect to the target tissue;
said second component of said delivery device is a tubular member; and
said second component of said cinch device is a cinch collar coupled to a distal end of said tubular member.

5. The system of claim 4, wherein a lumen defined within said cinch collar communicates with a lumen defined within said tubular member, and said proximally-extending portion of said compressive element extends through the lumens of said cinch collar and of said tubular member.

6. The system of claim 5, wherein said proximally-extending portion of said compressive element extends from the lumen of said tubular member through an opening in the wall of said tubular member to extend to said proximal portion of said compressive element outside the patient's body.

7. The system of claim 6, further comprising a cutter within said tubular member and movable to cut said proximally-extending portion of said compressive element.

8. The system of claim 1, wherein:
said distalmost end of said compressive element is coupled to a first component of said delivery device;
a section of said proximally-extending portion of said compressive element is movably coupled to a second component of said delivery device;
said first component of said delivery device is movable with respect to said second component of said delivery device to position said compressive element with respect to the target tissue; and
said proximal end of said compressive element is movable with respect to said delivery device to move said section of said compressive element with respect to said second component of said delivery device to adjust the size of said distal portion of said compressive element surrounding the target tissue.

9. The system of claim 8, further comprising:
a cinch element coupled to said first component of said delivery device, with said distalmost end of said compressive element coupled to said cinch element; and
a cinch collar coupled to said second component of said delivery device and movable with said second component of said delivery device and with respect to said first component of said delivery device to engage said cinch element to maintain a configuration of said distal portion of said compressive element.

10. The system of claim 1, further comprising an outer tubular delivery element defining a lumen through which said compression device is delivered to a target site within the patient's body.

11. The system of claim 10, further comprising a tension adjuster mounted on said outer tubular delivery element, wherein said proximal end of said compressive element is operably engaged with said tension adjuster, and movement of said tension adjuster moves said compressive element with respect to said delivery device.

12. The system of claim 10, further comprising a fitment mounted on said outer tubular delivery element and having at least one gripper configured to hold said proximal end of said compressive element in place with respect to said delivery device.

13. A tissue compression device comprising:
an elongated compressive element having a distalmost end, a flexible distal portion extending proximally from said distalmost end and arranged in a loop configuration, a proximally-extending portion extending proximally from said distal portion and terminating at a proximal end; and
a cinch device having a first component coupled to said distalmost end of said elongated compressive element;
wherein:
said distalmost end of said elongated compressive element and said first component of said cinch device are deliverable together to a target site within a body with said proximally-extending portion of said elongated compressive element extending to said proximal end of said elongated compressive element positioned outside the patient; and
said proximal end of said elongated compressive element is manipulable to adjust the loop configuration of said flexible distal portion of said elongated compressive element.

14. The tissue compression device of claim 13, wherein:
said cinch device further comprises a second component adapted to be delivered to the target site with said elongated compressive element and spaced apart from said first component of said cinch device such that said cinch device is delivered in an unlocked configuration; and
said first component and said second component of said cinch device are movable into engagement with each other to shift said cinch device into a locked configuration with respect to said elongated compressive element.

15. The tissue compression device of claim 14, wherein said proximally-extending portion of said elongated compressive element extends through an opening in said second component of said cinch device to form said distal portion of said elongated compressive element into the loop configuration, and is movable through the opening to adjust the loop configuration.

16. A method of applying and retaining a compressive element around target tissue within a patient, said method comprising:
delivering a distal portion of an elongated compressive element to a target site within the patient with a proximal end of the elongated compressive element remaining outside the patient;
surrounding target tissue with the distal portion of the elongated compressive element; and
maintaining a distalmost end of the elongated compressive element adjacent the surrounded target tissue while moving a proximally-extending portion of the elongated compressive element to adjust the area enclosed by and within the distal portion.

17. The method of claim 16, further comprising utilizing a first component of a delivery device used to deliver the distal portion of the elongated compressive element to maintain the distalmost end of the elongated compressive element in place with respect to the target tissue.

18. The method of claim 17, further comprising coupling a section of the proximally-extending portion of the elongated compressive element with respect to a second component of the delivery device to maintain the distal portion of the elongated compressive element in place surrounding the target tissue.

19. The method of claim 16, further comprising delivering the distalmost end of the elongated compressive element to the target tissue coupled to a first component of a cinch device.

20. The method of claim 19, further comprising delivering a second component of the cinch device with the proximally-extending portion of the elongated compressive element extending through an opening therethrough.
